(19) United States
(12) Patent Application Publication
(10) Pub. No.: US 2009/0306607 A1
(43) Pub. Date: Dec. 10, 2009

(54) Moxibustion device

(75) Inventor: Sakamoto Yasuhiro, Tokyo (JP)

Correspondence Address: EDWARDS ANGELL PALMER & DODGE LLP, P.O. BOX 55874, BOSTON, MA, 02205, US

(73) Assignee: Super Onnetsu Co. Ltd, Tokyo (JP)

(21) Appl. No.: 11/919,508

(22) PCT Filed: Apr. 21, 2006
(86) PCT No.: PCT/JP2006/308915

§ 371 Date: Oct. 29, 2007

(30) Foreign Application Priority Data
Apr. 28, 2005 (JP) 2005-002739
Jul. 11, 2005 (JP) 2005-201591

(51) Int. Cl. A61M 35/00; A61F 7/00

(52) U.S. Cl. 604/291

(57) ABSTRACT

A flat iron-like moxibustion device provided with a flat iron-like main body 2, a heater 5, a dome-like transparent glass cover 6 an outwardly curved outer surface that is brought into contact with a human body and an inwardly curved inner surface on which fine irregularity 6b is formed, a far infrared ray radiation layer 7 coated on the inner surface of the glass cover 6 on which fine irregularity 6b is formed, and a clay-like far infrared ray radiation material 8 filled into the inside of the glass cover 6 as being in close contact with the far infrared ray radiation layer 7, the moxibustion device being capable of substantially uniform far infrared ray radiation and having an excellent appearance.

### TECHNICAL FIELD

This invention relates to a moxibustion device in the form of a flat iron for performing warming and far infrared ray irradiation of a human body part.

### RELATED ART

As a moxibustion device in the form of a flat iron for performing warming and far infrared ray irradiation of a human body part, the one disclosed in Patent Publication 1 (International Publication No. WO2004/075986A1) has been known. Patent Publication 1 discloses a flat iron type moxibustion device that generates a far infrared ray by heating a far infrared ray radiation material containing a radon-generating rare native element or two or more of tourmaline ore, carbon, and a radon-generating rare native element and brings the far infrared ray to a deep part of a human body. Disclosed in FIGS. 1 and 2 of Patent Publication 1 is a structure wherein a far infrared ray radiation layer formed of a tourmaline ore layer, a carbon layer, and a radon-generating rare native element layer is laminated on an outer surface, which is an outwardly curved surface, of a dome-like metal or glass cover heated by a heater, and a far infrared ray radiation layer is coated or baked on an inwardly curved surface of a glass cover or the like. Disclosed in FIG. 8 of Patent Publication 1 are a structure wherein a far infrared ray radiation material is embedded under an inwardly curved surface of a glass cover and heated with a heater and a structure wherein a far infrared ray radiation layer is kept at 50° C., 60° C., 70° C., and 80° C., for example.

As technologies relating to warming and far infrared ray irradiation of a human body part, Patent Publications 2 to 5 have been known. Patent Publication 2 (JP-UM-A-2-141445 full text) discloses a far infrared ray radiation massage device that has a built-in vibration generation unit disposed inside a cylindrical body and is provided with a far infrared ray radiation layer obtained by coating an outer surface of an upper peripheral wall with ceramic particles such as alumina, magnesia, and zirconium and a cover to whose lower surface a heat generator is integrally fixed.

Patent Publication 3 (JP-UM-B-63-18156) discloses an electrical heating device obtained by: forming a sheet-like or plate-like substrate by mixing fine particles of natural radiogenic rare native elements such as samarskite, fergusonite, xenotime, thorogummite, modified zirconium with electroconductive carbon; providing each of opposite ends of the substrate with a nichrome wiring; and providing the nichrome wirings with a power source connection member.

Patent Publication 4 (JP-UM-B-3-25800) discloses a heat storing stimulation device that absorbs heat energy from a human body and converts the absorbed energy into a far infrared ray to radiate the infrared ray to the human body and a structure wherein: a surface of a colored ceramic to be brought into contact with the human body is coated with a glassy smooth thin film; a surface of the thin film is made smooth by softening and melting by baking; and the thin film is colored with carbon by reduction baking.

Patent Publication 5 (JP-A-2000-308668) discloses a structure wherein: a metal inner cylinder into which moxa is to be inserted is provided inside a heat insulating outer cylinder; a heat emission body that is formed of an outwardly curved metal mesh and a glass fiber cloth attached to an inner surface of the metal mesh is fitted to a tip of the inner cylinder; and a helical spring is provided at the tip of the inner cylinder, a cloth to which a ceramic powder and the like is deposited being attached to a tip of the spring.

As other known technologies relating to this invention, Patent Publication 6 (JP-A-2004-307313) discloses a ceramic production method with which a ceramic is obtained by: heating a powder of a flower of sulfur containing natural radium of Tamagawa hot spring of Akita prefecture to eliminate the sulfur component; mixing the sulfur flower powder with a powder obtained by heating a clay; adding moisture to the mixed powder to mold the mixed powder into a spherical shape or a plate-like shape, followed by drying; and heating at a high temperature. Patent Publication 6 also discloses that the obtained ceramic continuously radiates radium emanation and negative ions.

### DISCLOSURE OF THE INVENTION

In the flat iron-like moxibustion device of Patent Publication 1, in the case of forming the laminated far infrared ray radiation layer on the surface of the dome-like glass cover, bubbles are generated in the far infrared ray radiation layer, and fixation between the far infrared ray radiation layer that is formed by coating or baking and the glass cover surface is not sufficient. Therefore, problems that a clearance is formed in the far infrared ray radiation layer to cause fluctuation in far infrared ray radiation and that an appearance is deteriorated due to the clearance of far infrared ray radiation layer that can be seen through the transparent glass cover have been raised. Also, in the case of filling the far infrared ray radiation material into the inside of the glass cover, the far infrared ray radiation material moves inside the glass cover to slant, thereby raising the same problems that fluctuation in far infrared ray radiation is caused and that an appearance is deteriorated due to the clearance of far infrared ray radiation layer that can be seen through the transparent glass cover. Accordingly, there has been a demand for a flat iron-like moxibustion device capable of substantially uniform far infrared ray radiation and having an excellent appearance.

This invention is proposed in view of the above problems, and an object thereof is to provide a flat iron-like moxibustion device capable of substantially uniform far infrared ray radiation and having an excellent appearance.

A moxibustion device of this invention is characterized by comprising a flat iron-like main body, a heater provided at a front part of the main body, a dome-like transparent far infrared ray transmitting cover disposed at an outside of the heater and having an outwardly curved outer surface that is brought into contact with a human body and an inwardly curved inner surface on which a fine irregularity is formed, a far infrared ray radiation layer coated on the inner surface of the far infrared ray transmitting cover on which the fine irregularity is formed, and a clay-like far infrared ray radiation material to be filled into an inside of the far infrared ray transmitting cover while closely contacting with the far infrared ray radiation layer.

The moxibustion device of this invention is characterized in that an output from the heater is adjusted to an output corresponding to a heater temperature that keeps a temperature of the outer surface of the far infrared ray transmitting cover within a lower range of 37° C. to 43° C. and an output corresponding to a heater temperature that keeps a temperature of the outer surface of the far infrared ray transmitting cover within a higher range of 65° C. to 75° C.

The moxibustion device of this invention is characterized in that the heater output is adjustable and that a control unit reduces the heater output and simultaneously activates a cooling fan in response to an input for changing the heater output from the high temperature to the low temperature and stops the cooling fan in response to a heater temperature detection corresponding to the temperature by a temperature detection unit such as a thermistor.

The moxibustion device of this invention is characterized in that the cooling fan is provided at a rear of the heater at a position substantially corresponding to a first lateral end of a heater heat generating surface and that an air inlet is formed on the main body at a position substantially corresponding to a second lateral end that is substantially opposite to the first lateral end of the heater heat generating surface. For example, the first lateral end is a front end or a rear end of the heater heat generating surface, and the second lateral end is the rear end or the front end and the like of the heater heat generating surface.

The moxibustion device of this invention is characterized in that the far infrared ray transmitting cover is formed from a material having low heat conductivity. For example, the far infrared ray transmitting cover is a transparent glass cover or resin cover transmitting a far infrared ray.

The moxibustion device of this invention is characterized in that a space is defined between the heater and the far infrared ray radiation material.

The moxibustion device of this invention is characterized by in that the heat generating surface is brought into close contact with the far infrared ray radiation material.

The moxibustion device of this invention includes those obtained by adding a specific item to each of the inventions, modifying a part of a specific item of each of the inventions to another specific item, or deleting the specific item from the specific item of each of the inventions to a degree that a partial effect is achieved.

Since the far infrared ray radiation layer is coated on the inner surface of the far infrared ray transmitting cover on which the fine irregularity is formed, the moxibustion device of this invention is capable of preventing detachment of the far infrared ray radiation layer by the fixation with a high strength of the far infrared ray radiation layer to the inner surface owing to engagement with the fine irregularity. Further, since the clay-like far infrared ray radiation material is filled into the inside of the cover as being in close contact with the far infrared ray radiation layer, it is possible to dispose the far infrared ray radiation material at a position where bubbles have been generated or at a position where a partial detachment has occurred in the far infrared lay radiation layer. Also, since the close contact strength between the clay-like far infrared ray radiation material and the far infrared ray radiation layer is high, it is possible to stably dispose the far infrared ray radiation material. Therefore, it is possible to reliably dispose the far infrared ray radiation layer or the radiation material over substantially whole surface of the far infrared ray transmitting cover; to achieve substantially uniform far infrared ray radiation; to prevent the clearance from being seen from the transparent far infrared ray transmitting cover; and to improve the appearance of the moxibustion device. Further, it is possible to ensure the far infrared ray radiation layer and the radiation material each having a large surface area, thereby making it possible to effectively irradiate far infrared ray to a human body part.

By adjusting the temperature of the far infrared ray transmitting cover outer surface to the low temperature in the range of 37° C. to 43° C. and the high temperature in the range of 65° C. to 75° C., a user can use the moxibustion device continuously for a long time without receiving too strong heat stimulation by keeping the temperate to the low temperature of 37° C. to 43° C., which is a little higher than a body temperature, in the case where the user uses the moxibustion device by stationary positioning the moxibustion device at a desired part of body. In the case of using the moxibustion device while moving the moxibustion device along the skin, it is possible to keep the temperature to the high temperature of 65° C. to 75° C. Thus, it is possible to respond to the stationary positioning usage and the moving usage of the user. Further, since a feeling temperature is lowered by about 20° C. to 30° C. in the case where the moxibustion device is used while being moved along the skin, the feeling temperatures of the stationary use and the moving use are made substantially identical to each other.

Also, when changing the heater temperature from the high temperature to the low temperature, it is possible to rapidly lower the heater temperature and the temperature of the outer surface of the far infrared ray transmitting cover by activating the cooling fan until a predetermined heater temperature is achieved

Also, by providing the cooling fan at the rear of the heater at a position substantially corresponding to a first lateral end of a heater heat generating surface as well as by forming an air inlet on the main body at a position substantially corresponding to a second lateral end that is substantially opposite to the first lateral end of the heater heat generating surface, it is possible to form an air flow along a back surface of the heater, and it is possible to cool down by depriving heat of the heater by the air flow.

By forming the far infrared ray transmitting cover from a material reduced in heat conductivity, it is possible to lower the heat conductivity of the heat of the heater, thereby suppressing excessive heat stimulation and sharp heat stimulation to a human body part.

Also, by forming the space between the heater and the far infrared ray radiation material, it is possible to reduce the heat conductivity as well as to suppress excessive heat stimulation and sharp heat stimulation to a human body part.

Also, since the heat generating surface of the heater is brought into close contact with the far infrared ray radiation material, it is possible to transmit the heat to the far infrared ray radiation material, the far infrared ray radiation layer, and the far infrared ray transmitting cover, thereby reducing electrical power consumption.

### BRIEF DESCRIPTION OF THE DRAWINGS

FIG. 1(a) is a front view showing a moxibustion device according to a first embodiment;

FIG. 1(b) is a plan view showing the moxibustion device according to the first embodiment;

FIG. 2 is a vertical sectional view showing the moxibustion device according to the first embodiment;

FIG. 3 is a vertical sectional view showing a lamination structure of a glass cover, a far infrared ray radiation layer, and a far infrared ray radiation material in the moxibustion device according to the first embodiment;

FIG. 4 is a block diagram showing a control structure of the moxibustion device according to the first embodiment; and

FIG. 5 is a vertical sectional view showing an arrangement of a glass cover, a far infrared ray radiation layer, and a far infrared ray radiation material in a moxibustion device according to a second embodiment.

### BEST MODE FOR CARRYING OUT THE INVENTION

A first embodiment of a moxibustion device of this invention will be described.

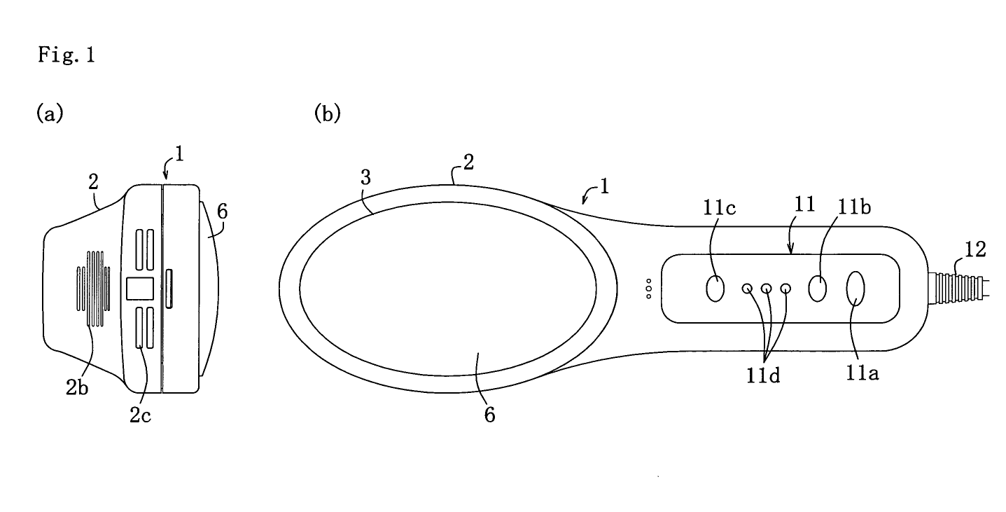

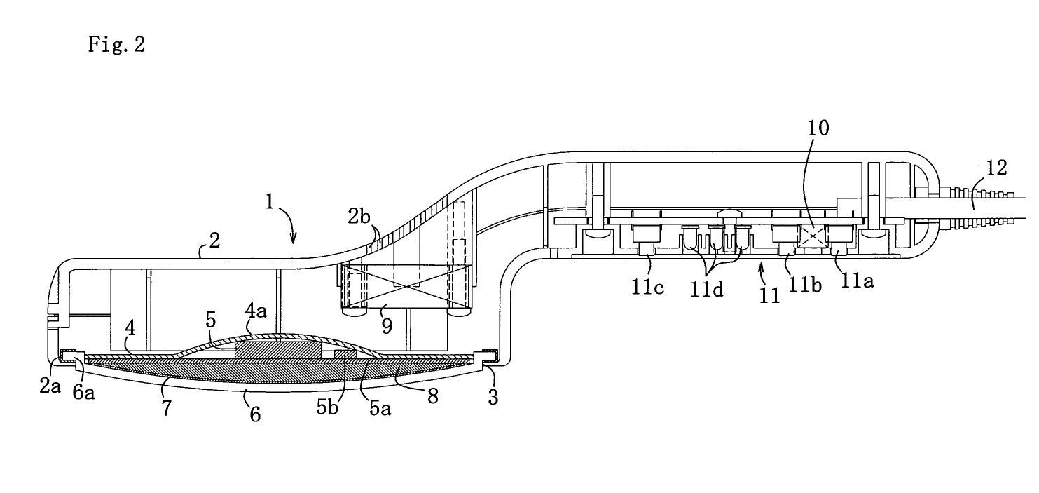

As shown in FIGS. 1 to 3, a flat iron-like moxibustion device 1 of the first embodiment has a flat iron-like main body 2 that is substantially in the form of a scoop in a plan view and an oval opening 3 that is formed at a front part of the main body 2. Inside the opening 3, a heat insulating plate 4 having a shape and a size that are substantially the same as those of the opening 3 in a plan view and a recess 4a formed at a central part is provided as being fixed to the opening 3, and a heater 5 having a flat plate-like heat generation surface 5a having a shape that is substantially the same as that of the opening 3 in a plan view and a size that is a little smaller than the opening 3 is housed in the recess 4a of the heat insulating plate, the heat generating surface 5a being disposed at a front surface of the heat insulating plate 4. Denoted by 5b is a thermistor provided in the heater 5.

In front of the heat generating surface 5a of the heater 5, a dome-like glass cover 6 having an oval shape in a plan view is provided with a space being defined between the heat generating surface 5a and the glass cover 6 as a transparent far infrared ray transmitting cover of which an outwardly curved outer surface is to be brought into contact with a human body part, and the glass cover 6 is disposed in such a manner that an engagement piece 6a which is formed on a circumference when the outwardly curved surface is at the front is engaged with a circular engagement part 2a disposed along a circumference of the opening 3 of the main body 2 and having a lateral U-shape in a sectional view. Attachment of the glass cover 6 to the opening 3 that is realized by the engagement of the engagement piece 6a bended into an L-shape with the engagement part 2a is preferred since such attachment is good in stability and free from necessity of sealing. However, a structure of fitting a circumference of a glass cover having substantially oval shape that does not have the engagement piece 6a into an opening of a main body while orienting an outwardly curved surface to the front and sealing the fitting portion with packing may also be employed, for example. The far infrared ray transmitting cover of this invention may preferably be formed from a material that is reduced in heat conductivity, and a resin cover having low heat conductivity and the like, for example, may be used in place of the glass cover 6.

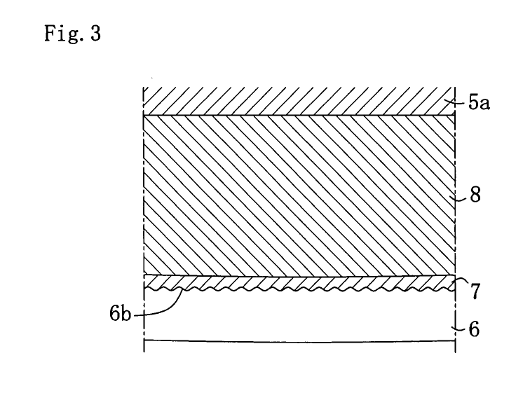

On an inner surface of the inwardly curved surface of the glass cover 6, fine irregularity 6b is formed by sand blast or the like as shown in FIG. 3. A far infrared ray radiation layer 7 is coated and baked on or welded to the inner surface of the glass cover 6 on which the fine irregularity 6b is formed, and the far infrared ray radiation layer 7 is fixed with a high strength as being engaged with the fine irregularity 6b formed on the inner surface of the glass cover 6. A clay-like far infrared ray radiation material 8 is filled into the inside of the inwardly curved surface of the glass cover 6 to be embedded on the far infrared ray radiation layer 7. Since the far infrared ray radiation material 8 is filled with an upper surface thereof contacting with the heater heat generating surface 5a as well as the far infrared ray radiation layer 7 that is high in friction coefficient, the far infrared ray radiation material 8 is prevented from being moved or slanted under the inwardly curved surface of the glass cover 6, thereby achieving highly stable placement. Apart from the simple close contact placement, a structure of bringing the far infrared ray radiation material 8 and the heat generating surface 5a into close contact with each other by using a highly heat conductive adhesive agent or the like may be employed.

Appropriate materials may be used for the far infrared ray radiation layer 7 and the clay-like far infrared ray radiation material 8, such as a ceramic including alumina, magnesia, zirconium, and the like; a natural radiogenic rare native element including tourmaline, thorium, radon, and the like; and carbon, which are used alone or in combination of two or more. Further, the ceramic of Patent Publication 6 obtained by heating a powder of a flower of sulfur containing natural radium to eliminate the sulfur component; mixing the sulfur flower powder with a powder obtained by heating a clay; adding moisture to the mixed powder to mold the mixed powder into a spherical shape or a plate-like shape, followed by drying; and heating at a high temperature may be mixed.

In the case of forming the far infrared ray radiation layer 7 on the inner surface of the glass cover 6 by coating, 1.5 wt % of a mineral ore having a far infrared ray radiation effect and 98.5 wt % of a heat conductive cement are mixed and kneaded; the kneaded substance is coated on the inner surface of the glass cover 6 followed by eliminating bubbles by vacuum suction in a vacuum desiccator; the coating material is pressed for defoaming filling, followed by standing for about 3 to 5 hours; and pores are formed on a surface of the semi-solid cement by using an awl, followed by natural drying for 1 to 2 days. After that, the glass cover 6 and the coating substance that are compressed by using a dedicated tool are placed in a drying device; a temperature of the drying device is raised to 120° C. in 2 hours, followed by maintaining 120° C. for 5 hours or more and standing in the air; and the glass cover is stored in a sealed container with a desiccant. The glass cover 6 is formed by performing the above-described process steps and the like. A mixing ratio of the mineral ore having far infrared ray radiation effect to the heat conductive cement may preferably be set in the range of 0.1 to 10 wt %: 99.9 to 90 wt %.

At the rear of the heat insulating plate 4 and the heater 5 in the main body 2, a cooling fan 9 is provided at a position corresponding to a substantially rear end of the opening 3 and the heater heat generating surface 5a, and a plurality of air outlets 2b each in the form of a slit are formed on the main body positioned at the rear of the cooling fan 9. Air inlets 2c each in the form of a slit is formed on a front end lateral surface of the main body 2. By activating the cooling fan 9, the air heated inside the main body 2 due to the heating by the heater 5 is discharged from the air outlets 2b simultaneously with an intake of the outside air into the main body 2 from the inlets 2c. An air flow from the air inlets 2c positioned at the substantially front end of the heater heating surface 5a to the cooling fan 9 and the discharge outlets 2b positioned at the substantially rear end is formed along the heater heating surface 5a, and the air flow cools down the heater heat generating surface 5a and the like by effectively depriving the heat.

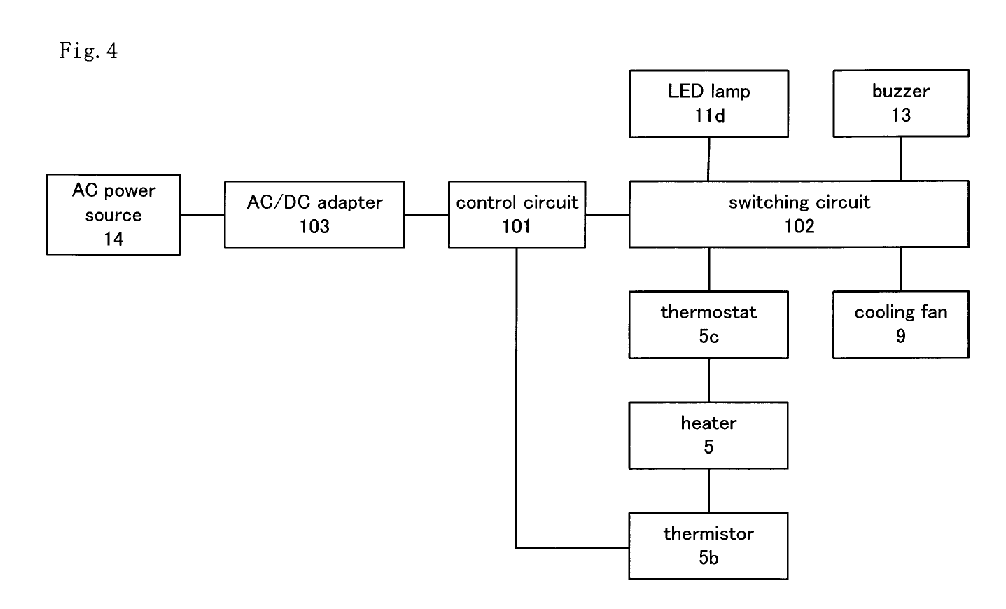

Inside a handle of the main body 2, a circuit housing part 10 is disposed. The circuit housing part 10 houses a control circuit 101 formed of a CPU and a memory wherein the CPU executes a predetermined processing in accordance with a control program stored in the memory, a switch circuit 102, and an AC/DC adapter 103 (see FIG. 4). As shown in FIG. 4, the switching circuit 102 performs switching of ON/OFF and operation states of the heater 5 connected thereto via a thermostat 5c for retaining a temperature of the heater to a predetermined value, the cooling fan 9, LED lamps 11d described later in this specification, a buzzer 13 (not shown in FIGS. 1 to 3) beeping at a predetermined pattern when a predetermined time has elapsed from the start of use and when the buttons 11a to 11c described later in this specification are pushed according to control of the control circuit 101.

An operation input part 11 is provided at a front surface of the handle of the main body 2, and, on the operation input part 11, the power source button 11a, the low temperature mode button 11b for setting a temperature of the heater heat generating surface 5a to a low temperature mode by which a temperature of the outer surface of the glass cover 6 is kept at 37° C., 40° C., or 43° C., the high temperature mode button 11c for setting a temperature of the heater heat generating surface 5a to a high temperature mode by which a temperature of the outer surface of the glass cover 6 is kept at 65° C., 70° C., or 750C, and the LED lamps 11d of which red light/green light is selected to be lit/extinguished are provided. Input via each of the buttons 11a to 11c is recognized by the control circuit 101, so that the control circuit 101 outputs a predetermined control instruction to the switching circuit 102. Denoted by 12 is a power source cord for supplying a power source by an alternating current power source 14 such as a household electric outlet to the AC/DC adapter 103.

When using the moxibustion device 1 of the above-described embodiment, power is turned on by pushing the power source button 11a for about one second, and the control circuit 101 controls the switching circuit 102 to allow the heater 5 to output at a predetermined temperature of the heater 5 corresponding to the outer surface temperature of 37° C. of the glass cover 6, e.g. at 47° C., in response to the input via the power source button 11a, so that the switching circuit 102 brings the heater 5 into the ON state at the predetermined temperature. Further, the control circuit 101 detects the predetermined temperature by comparing a measurement value inputted from the thermistor 5b with the predetermined temperature and controls the switching circuit 102 in response to the detection to cause the LED lamp 11d positioned at a lower part to emit green light (low temperature state in low temperature mode). Due to the output of the heater 5, the far infrared ray radiation material 8, the far infrared ray radiation layer 7, and the glass cover 6 that contacts with the heater heat generating surface 5a are heated, so that a human body part contacting the outer surface of the glass cover 6 is warmed and that the far infrared ray is radiated to the human body part.

Subsequently, in response to a temperature change input performed by pushing the high temperature mode button 11c for about one second, the control circuit 101 controls the switching circuit 102 so that the heater 5 outputs at a predetermined temperature of the heater 5 corresponding to the outer surface temperate of 65° C. of the glass cover 6, e.g. at 75° C., and the switching circuit 102 changes the output from the heater 5 to a state of the predetermined temperature. Further, the control circuit 101 detects the predetermined temperature by comparing a measurement value inputted from the thermistor 5b with the predetermined temperature and controls the switching circuit 102 in response to the detection to cause the LED lamp 11d positioned at the lower part to emit red light (low temperature state in high temperature mode).

Subsequently, in response to a temperature change input performed by pushing the high temperature mode button 11c for about one second, the control circuit 101 controls the switching circuit 102 so that the heater 5 outputs at a predetermined temperature of the heater 5 corresponding to the outer surface temperate of 70° C. of the glass cover 6, e.g. at 80° C., and the switching circuit 102 changes the output from the heater 5 to a state of the predetermined temperature. Further, the control circuit 101 detects the predetermined temperature by comparing a measurement value inputted from the thermistor 5b with the predetermined temperature and controls the switching circuit 102 in response to the detection to cause the two LED lamps 11d positioned at the lower part and an intermediate part to emit red light (intermediate temperature state in high temperature mode).

Subsequently, in response to a temperature change input performed by pushing the high temperature mode button 11c for about one second, the control circuit 101 controls the switching circuit 102 so that the heater 5 outputs at a predetermined temperature of the heater 5 corresponding to the outer surface temperate of 75° C. of the glass cover 6, e.g. at 85° C., and the switching circuit 102 changes the output from the heater 5 to a state of the predetermined temperature. Further, the control circuit 101 detects the predetermined temperature by comparing a measurement value inputted from the thermistor 5b with the predetermined temperature and controls the switching circuit 102 in response to the detection to light the three red LED lamps 11d positioned at the lower part, the intermediate part, and an upper part (high temperature state in high temperature mode). The control circuit 101 executes a processing for changing the temperature to the low temperature in the high temperature mode when a temperature change input is performed again by pushing the high temperature mode button 11c, i.e. the control circuit 101 executes a processing for changing the temperature in a loop of the low temperature state, the intermediate temperature state, and the high temperature state in the high temperature mode every time a temperature change input is performed by pushing the high temperature mode button 11c for about one second. Thus, it is possible to perform the temperature settings in the stepwise manner in the high temperature mode.

Also, in response to an input for changing the temperature from the low temperature state in the low temperature mode performed by pushing the low temperature mode button 11b for about one second, the control circuit 101 controls the switching circuit 102 so that the heater 5 outputs at a predetermined temperature of the heater 5 corresponding to the outer surface temperate of 40° C. of the glass cover 6, e.g. at 50° C., and the switching circuit 102 changes the output from the heater 5 to a state of the predetermined temperature. Further, the control circuit 101 detects the predetermined temperature by comparing a measurement value inputted from the thermistor 5b with the predetermined temperature and controls the switching circuit 102 in response to the detection to cause the two LED lamps 11d positioned at the lower part and the intermediate part to emit green light (intermediate temperature state in low temperature mode).

Subsequently, in response to an input for changing the temperature performed by pushing the low temperature button 11b for about one second, the control circuit 101 controls the switching circuit 102 so that the heater 5 outputs at a predetermined temperature of the heater 5 corresponding to the outer surface temperate of 43° C. of the glass cover 6, e.g. at 53° C., and the switching circuit 102 changes the output from the heater 5 to a state of the predetermined temperature. Further, the control circuit 101 detects the predetermined temperature by comparing a measurement value inputted from the thermistor 5b with the predetermined temperature and controls the switching circuit 102 in response to the detection to cause the three LED lamps 11d positioned at the lower part, the intermediate part, and the upper part to emit green light (high temperature state in low temperature mode). The control circuit 101 executes a processing for changing the temperature to the low temperature in the low temperature mode when a temperature change input is performed again by pushing the low temperature mode button 11b, i.e. the control circuit 101 executes a processing for changing the temperature in a loop of the low temperature state, the intermediate temperature state, and the high temperature state in the low temperature mode every time a temperature change input is performed by pushing the low temperature mode button 11b. Thus, it is possible to perform the temperature settings in the stepwise manner in the low temperature mode.

In the case of shifting from the high temperature state to the low temperature state in the above-described temperature change, the control circuit 101 controls the switching circuit 102 in response to the temperature change input to activate the cooling fan 9 and detects the predetermined temperature by comparing a measurement value inputted from the thermistor 5b with the predetermined low temperature to stop the cooling fan 9 by controlling the switching circuit 102 in response to the detection. With the above-described structure, it is possible to rapidly shift from the high temperature state to the low temperature state. Alternatively, a structure of flashing the red or green LED lamp 11d at the temperature shift by the control on the switching circuit 102 by the control circuit 101, a structure of beeping the buzzer 13 at a predetermined pattern at the temperature shift by the control on the switching circuit 102 by the control circuit 101, or the like may be utilized.

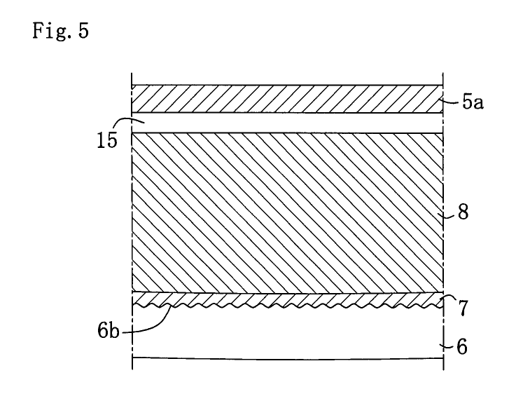

Though the heat generating surface 5a of the heater 5 is in close contact with the upper surface of the far infrared ray radiation material 8 to effectively transmit the heat to a human body part via the far infrared ray radiation material 8, the far infrared ray radiation layer 7, and the glass cover 6 and to achieve a reduction in electric power consumption in the first embodiment, a space 15 may be defined between the upper surface of the far infrared ray radiation material 8 and the heater heat generating surface 5a as described in a second embodiment of FIG. 5. In the structure of defining the space 15, since the heat from the heater heat generating surface 5a is transmitted to the far infrared ray radiation material 8 via the air that is low in heat conductivity and then to a human body part contacting the outer surface of the glass cover 6 via the far infrared ray radiation layer 7 in the form of a thin film and the glass cover 6 having a low heat conductivity, it is possible to prevent a sharp heat stimulation to the human body part.

It is preferable to keep a height of the space 15 between the heater heat generating surface 5a and the far infrared ray radiation material 8, i.e. a thickness of the air layer, to a substantially constant height by, in the same manner as in the first embodiment, providing the film-like far infrared ray radiation layer 7 curved along the inner surface of the substantially oval and dome-like glass cover 6 and providing the far infrared ray radiation material 8 on the far infrared ray radiation layer 7 in such a manner as to fill up the far infrared ray radiation layer 7 and the substantially oval and dome-like recessed part of the glass cover 6, since the substantially constant height makes it possible to transmit a substantially uniform heat amount to the entire far infrared ray radiation material 8 and the far infrared ray radiation layer 7. It is possible to realize such uniform heat transmission also by forming the far infrared ray radiation material 8 as a film curved along the shape of the oval and dome-like inwardly curved surfaces of the far infrared ray radiation layer 7 and the glass cover 6 or as an inwardly curved layer like the far infrared ray radiation layer 7 to define the space 15 that is varied in height between the flat plate-like heat generating surface 5a and the inwardly curved surface of the far infrared ray radiation material 8, thereby varying the height of the space 15 and an air layer thickness of the space 15.

Alternatively, in the case of forming the far infrared ray radiation material 8 as the inwardly curved surface, a structure of forming the heat generating surface 5a as an inwardly curved surface to fit the shape of the far infrared ray radiation material 8 and providing the far infrared ray radiation material 8 and the heat generating surface 5a with the inwardly curved surfaces being in close contact with each other or a structure of providing the space 15 having a height and a air layer thickness that is substantially constant or varied between the inwardly curved surfaces of the far infrared ray radiation material 8 and the heat generating surface 5a may be employed.

### INDUSTRIAL APPLICABILITY

This invention is utilized as a moxibustion device for heating a human body part while being pressed against the shoulder, for example, or being moved along the skin to irradiate the part with a far infrared ray.

1. A flat iron-like moxibustion device characterized by comprising: a flat iron-like main body, a heater provided at a front part of the main body, a dome-like transparent far infrared ray transmitting cover disposed at an outside of the heater and provided with an outwardly curved outer surface that is brought into contact with a human body and an inwardly curved inner surface on which a fine irregularity is formed, a far infrared ray radiation layer coated on the inner surface of the far infrared ray transmitting cover on which the fine irregularity is formed, and a clay-like far infrared ray radiation material to be filled into an inside of the far infrared ray transmitting cover while closely contacting with the far infrared ray radiation layer.

2. The moxibustion device according to claim 1, characterized in that an output from the heater is adjusted to an output corresponding to a heater temperature that keeps a temperature of the outer surface of the far infrared ray transmitting cover within a lower range of 37° C. to 43° C. and an output corresponding to a heater temperature that keeps a temperature of the outer surface of the far infrared ray transmitting cover within a higher range of 65° C. to 75° C.

3. The moxibustion device according to claim 1, characterized in that the heater output is adjustable and that a control unit reduces the heater output and simultaneously activates a cooling fan in response to an input for changing the heater output from the high temperature to the low temperature and stops the cooling fan in response to a heater temperature detection corresponding to the temperature by a temperature detection unit.

4. The moxibustion device according to claim 2, characterized in that the cooling fan is provided at a rear of the heater at a position substantially corresponding to a first lateral end of a heater heat generating surface and that an air inlet is formed on the main body at a position substantially corresponding to a second lateral end that is substantially opposite to the first lateral end of the heater heat generating surface.

5. The moxibustion device according to claim 1, characterized in that the cooling fan is provided at a rear of the heater at a position substantially corresponding to a first lateral end of a heater heat generating surface and that an air inlet is formed on the main body at a position substantially corresponding to a second lateral end that is substantially opposite to the first lateral end of the heater heat generating surface.

6. The moxibustion device according to claim 2, characterized in that cooling fan is provided at a rear of the heater at a position substantially corresponding to a first lateral end of a heater heat generating surface and that an air inlet is formed on the main body at a position substantially corresponding to a second lateral end that is substantially opposite to the first lateral end of the heater heat generating surface.

7. The moxibustion device according to claim 3, characterized in that the cooling fan is provided at a rear of the heater at a position substantially corresponding to a first lateral end of a heater heat generating surface and that an air inlet is formed on the main body at a position substantially corresponding to a second lateral end that is substantially opposite to the first lateral end of the heater heat generating surface.

8. The moxibustion device according to claim 4, characterized in that the cooling fan is provided at a rear of the heater at a position substantially corresponding to a first lateral end of a heater heat generating surface and that an air inlet is formed on the main body at a position substantially corresponding to a second lateral end that is substantially opposite to the first lateral end of the heater heat generating surface.

9. The moxibustion device according to claim 1, characterized in that the far infrared ray transmitting cover is formed from a material having low heat conductivity.

10. The moxibustion device according to claim 2, characterized in that the far infrared ray transmitting cover is formed from a material having low heat conductivity.

11. The moxibustion device according to claim 3, characterized in that the far infrared ray transmitting cover is formed from a material having low heat conductivity.

12. The moxibustion device according to claim 4, characterized in that the far infrared ray transmitting cover is formed from a material having low heat conductivity.

13. The moxibustion device according to claim 5, characterized in that the far infrared ray transmitting cover is formed from a material having low heat conductivity.

14. The moxibustion device according to claim 6, characterized in that the far infrared ray transmitting cover is formed from a material having low heat conductivity.

15. The moxibustion device according to claim 7, characterized in that the far infrared ray transmitting cover is formed from a material having low heat conductivity.

16. The moxibustion device according to claim 8, characterized in that the far infrared ray transmitting cover is formed from a material having low heat conductivity.

17. The moxibustion device according to claim 1, characterized in that a space is defined between the heater and the far infrared ray radiation material.

18. The moxibustion device according to claim 17, characterized in that the space has a height that is substantially constant.

19. The moxibustion device according to claim 1, characterized in that the heat generating surface of the heater is brought into close contact with the far infrared ray radiation material.
